(19) United States
(12) Patent Application Publication
(10) Pub. No.: US 2022/0378589 A1
(43) Pub. Date: Dec. 1, 2022

(54) STENT, STENT PRECURSOR PRODUCTION DEVICE, AND STENT PRODUCTION METHOD

(71) Applicant: IR MEDICAL ATELIER CO., LTD., Koriyama-shi, Fukushima-ken (JP)

(72) Inventor: Daisuke KAWABE, Koriyama-shi, Fukushima-ken (JP)

(21) Appl. No.: 17/776,350

(22) PCT Filed: Nov. 12, 2020
(86) PCT No.: PCT/JP2020/042188

§ 371 Date: May 12, 2022

(30) Foreign Application Priority Data
Nov. 13, 2019 (JP) 2019-205585

(51) Int. Cl. A61F 2/88; A61B 17/12

(52) U.S. Cl. CPC A61F 2/88; A61B 17/12118; A61B 2017/00867

(57) ABSTRACT

In this stent, two superelastic fine wires are disposed along the axial direction at a prescribed helical pitch so as to have a prescribed stent inner diameter D0, while a pair is formed between two helical fine wires that are disposed across a micro gap of a size not more than five times the wire diameter of the fine wires in such a manner as to include a mutually contacting state. A prescribed reticulation gap is formed by crossing a clockwise-wound helical fine wire pair and a counterclockwise-wound helical fine wire pair in a plain-woven fashion, so as to have an axial gap equal to [(prescribed helical pitch)−{2×(fine wire diameter)}−(micro gap)] and a circumferential gap equal to [{(stent inner circumferential length corresponding to stent inner diameter)/N}−{2×(fine wire diameter)}−(micro gap

### TECHNICAL FIELD

The present disclosure relates to a stent, a stent precursor production device, and a stent production method, and particularly relates to a coil-assisted stent having a stitch gap of a size through which a microcatheter for guiding a coil placed in a vascular aneurysm can pass, as well as its precursor production device and its production method.

### BACKGROUND

As a treatment of a vascular aneurysm, which is a disease of an artery or a vein, there are known a coil placed inside the vascular aneurysm while using a catheter, a balloon, or the like in combination, a stent placed in the main vessel of a blood vessel having a vascular aneurysm, and the like.

Recently, stent combination coil embolization in which a stent is placed in a blood vessel to prevent the coil from jumping out has also been performed. In this case, the stent is placed across the neck of the vascular aneurysm, and the microcatheter for guiding the coil is inserted toward the inside of the vascular aneurysm between the blood vessel wall and the stent (the jail method) or through the stitch gap of the stent (the transcell method).

Patent Document 1 describes an example in which, in a stent of a hose-shaped body having a plurality of superelastic thin wires knitted in a braid shape, the stent includes a thin wire dense hose portion having a fine stitch gap and a thin wire coarse hose portion having a coarse stitch gap. Here, it is described that the microcatheter used for coil embolization can be inserted from the inside of the stent to the outside of the stent through the coarse stitch gap of the thin wire coarse hose portion toward the inside of the vascular aneurysm.

### CITATION LIST

### Patent Literature

Patent Document 1: WO 2009/008373 A

### SUMMARY

### Technical Problem

A stent that is placed at the site of the vascular aneurysm and allows the microcatheter to pass from the inside to the outside of the hose-shaped body by the transcell method is called a coil-assisted stent. The coil-assisted stent needs to be placed at the site of the vascular aneurysm, have a blood vessel wall expansion force such that it is not swept away by the blood flow, and have a wide stitch gap through which the microcatheter can pass.

The stent placed in the blood vessel uses a superelastic hose-shaped body that memorizes a predetermined diameter. When the stent is inserted into the blood vessel, the stent is elongated in the axial direction, is released from the extension at the site of the vascular aneurysm and returns to the predetermined diameter and generates a blood vessel wall expansion force due to a difference between the predetermined diameter and the blood vessel inner diameter. Assuming that the conditions such as the blood vessel inner diameter, the predetermined diameter of the stent, and the diameter of the superelastic thin wires constituting the stent are the same, the blood vessel wall expansion force increases as the number of superelastic thin wires per unit area of the outer peripheral surface of the stent increases. On the other hand, the stitch gap of the superelastic hose-shaped body becomes larger as the number of superelastic thin wires per unit area of the outer peripheral surface of the stent decreases.

That is, if the stitch gap is set to be large such that the microcatheter can pass therethrough, the number of superelastic thin wires per unit area of the outer peripheral surface of the stent decreases, the blood vessel wall expansion force of the stent decreases, and the stent cannot be properly placed. On the other hand, when the blood vessel wall expansion force is set to be large, the number of superelastic thin wires per unit area of the outer peripheral surface of the stent increases, the interval between the adjacent superelastic thin wires is narrowed, the mesh gap is reduced, and the microcatheter cannot pass therethrough.

As a method for solving this conflicting relationship, it is conceivable to use superelastic thin wires made of a highly elastic material, but it is necessary to use a superelastic material having biocompatibility in order to place the wires in the human body. At the present time, thin wires using a nickel-titanium alloy called nitinol are common, and in the case of using other materials, it is necessary to confirm biocompatibility and the like, and the cost becomes remarkably high. Alternatively, it is conceivable to provide a special placing structure at both ends or the like of the stent, but the placing treatment becomes complicated, and the stent production process becomes complicated, resulting in an increase in cost.

Therefore, there is demand for a stent, a stent precursor production device, and a stent production method that improve the blood vessel wall expansion force while having the stitch gap of the same size under the same other conditions without using a special material or a placing structure.

### Solution to Problem

The stent according to the present disclosure includes: with one pair consisting of two spiral thin wires in which two thin wires having superelasticity have a predetermined stent inner diameter at a predetermined spiral pitch along an axial direction, and are disposed in a minute gap that includes a contact state with each other and is five times or less a wire diameter of the thin wires, N pairs of right-handed spiral thin wire pairs wound in a right-handed spiral shape; and N pairs of left-handed spiral thin wire pairs wound in a left-handed spiral shape, the stent having an axial gap of [(predetermined spiral pitch)−{2×(wire diameter of thin wire)}−(minute gap)] and a circumferential gap of [{(stent inner circumferential length corresponding to stent inner diameter)/N}−{2×(wire diameter of thin wire)}−(minute gap)] as a predetermined stitch gap formed by intersecting the right-handed spiral thin wire pair and the left-handed spiral thin wire pair in a plain weave shape.

According to the above configuration, the predetermined stitch gap is formed by intersecting N pairs of the right-handed spiral thin wire pairs and N pairs of the left-handed spiral thin wire pairs in a plain weave shape with two spiral thin wires disposed in the minute gap as one pair. As compared with the case where the predetermined stitch gap is formed by intersecting the N right-handed spiral thin wires and the N left-handed spiral thin wires in a plain weave shape, the number of spiral thin wires per unit area of the outer peripheral surface of the stent is doubled while the predetermined stitch gap is the same, in a manner that the blood vessel wall expansion force can be approximately doubled.

In the stent according to the present disclosure, the predetermined stitch gap preferably has a size through which a microcatheter that guides a coil placed in a cerebrovascular aneurysm can pass. According to the above configuration, the microcatheter can be disposed in the inner diameter of the stent and inserted into the vascular aneurysm through the microcatheter from the predetermined stitch gap.

The stent precursor production device according to the present disclosure includes: a main body housing portion having a cylindrical outer shape; a winding shaft movement mechanism that moves and drives a winding shaft at an axial movement speed along an axial direction of a central axis of the main body housing portion; a one-side traveling path and an other-side traveling path meandering around the central axis of the main body housing portion while intersecting each other in a substantially figure-eight shape on an upper surface of the main body housing portion and making one round in a circumferential shape; 4N bobbin carriers in which there are erected a bobbin shaft rotatably supporting a knitting yarn bobbin around which a thin wire made of a shape memory alloy is wound, and a yarn passing portion that applies a predetermined tension to the thin wire pulled out from the knitting yarn bobbin and guides the thin wire to a thin wire supply hole at a predetermined height position; with the central axis of the main body housing portion as a revolution axis, a bobbin carrier driving portion that drives 2N bobbin carriers disposed on the one-side traveling path among the 4N bobbin carriers to travel at a revolution speed clockwise around the revolution axis, and drives other 2N bobbin carriers disposed on the other-side traveling path to travel at the revolution speed counterclockwise around the revolution axis so as not to interfere with the 2N bobbin carriers traveling clockwise at a substantially figure-eight shaped intersection position; and a control unit that controls the revolution speed and the axial movement speed, in which the bobbin carriers include 2N pairs of bobbin carrier pairs disposed at a predetermined adjacent pair interval wider than a predetermined proximity interval as one pair of the bobbin carrier pair including two bobbin carriers disposed at the predetermined proximity interval determined in advance on the one-side traveling path and the other-side traveling path.

According to the above configuration, since the interval between the adjacent bobbin carriers can be set to different intervals of the predetermined proximity interval and the predetermined adjacent pair interval, the gap between the adjacent spiral thin wires can be set to different gaps also for the spiral thin wire formed by winding the thin wire from the bobbin carrier around the winding shaft using this.

In the stent production method according to the present disclosure, a stent precursor production device is used, the stent precursor production device including a main body housing portion having a cylindrical outer shape, a winding shaft movement mechanism that moves and drives a winding shaft at an axial movement speed along an axial direction of a central axis of the main body housing portion, a one-side traveling path and an other-side traveling path meandering around the central axis of the main body housing portion while intersecting each other in a substantially figure-eight shape on an upper surface of the main body housing portion and making one round in a circumferential shape, 4N bobbin carriers in which there are erected a bobbin shaft rotatably supporting a knitting yarn bobbin around which a thin wire made of a shape memory alloy is wound, and a yarn passing portion that applies a predetermined tension to the thin wire pulled out from the knitting yarn bobbin and guides the thin wire to a thin wire supply hole at a predetermined height position, with the central axis of the main body housing portion as a revolution axis, a bobbin carrier driving portion that drives 2N bobbin carriers disposed on the one-side traveling path among the 4N bobbin carriers to travel at a revolution speed clockwise around the revolution axis, and drives other 2N bobbin carriers disposed on the other-side traveling path to travel at the revolution speed counterclockwise around the revolution axis so as not to interfere with the 2N bobbin carriers traveling clockwise at a substantially figure-eight shaped intersection position, and a control unit that controls the revolution speed and the axial movement speed, in which the bobbin carriers include 2N pairs of bobbin carrier pairs disposed at a predetermined adjacent pair interval wider than a predetermined proximity interval as one pair of the bobbin carrier pair including two bobbin carriers disposed at the predetermined proximity interval determined in advance on the one-side traveling path and the other-side traveling path, the stent production method including disposing a knitting yarn bobbin in which the thin wire made of the shape memory alloy is wound on the bobbin shaft of each of the 4N bobbin carriers, in each of the 4N bobbin carriers, applying a predetermined tension to the thin wire pulled out from the knitting yarn bobbin to pull out the thin wire from the thin wire supply hole at the predetermined height position, winding each of distal ends of the pulled out 4N thin wires around the winding shaft having a predetermined outer diameter corresponding to the stent inner diameter, moving the winding shaft at a predetermined axial movement speed along an axial direction of the revolution axis while operating a carrier driving portion at a predetermined revolution speed, regarding N pairs of bobbin carrier pairs disposed on the one-side traveling path, driving the 2N bobbin carriers to travel clockwise at the revolution speed with respect to the revolution axis while maintaining the predetermined proximity interval and the predetermined adjacent pair interval along the one-side traveling path, and winding 2N thin wires pulled out from the thin wire supply hole of each of the 2N bobbin carriers around the winding shaft at a predetermined spiral pitch in a right-handed spiral shape to obtain 2N right-handed spiral thin wires having the stent inner diameter, and regarding N pairs of bobbin carrier pairs disposed on the other-side traveling path, driving the 2N bobbin carriers to travel counterclockwise at the revolution speed with respect to the revolution axis while maintaining the predetermined proximity interval and the predetermined adjacent pair interval along the other-side traveling path, and winding 2N thin wires pulled out from the thin wire supply hole of each of the 2N bobbin carriers around the winding shaft at a predetermined spiral pitch in a left-handed spiral shape to obtain 2N left-handed spiral thin wires having the stent inner diameter, obliquely intersecting each of the 2N right-handed spiral thin wires and each of the 2N left-handed spiral thin wires in a plain weave shape to knit a stent precursor of a hose-shaped body while forming a diamond-like stitch gap, removing the knitted stent precursor from the stent precursor production device together with the winding shaft, since in a state of being wound around the winding shaft, the 2N right-handed spiral thin wires include two right-handed spiral thin wires disposed at a predetermined proximity gap corresponding to the predetermined proximity interval as one pair of the right-handed spiral thin wire pair, and N pairs of right-handed spiral thin wire pairs as a predetermined adjacent pair gap corresponding to the predetermined adjacent pair interval, and the 2N left-handed spiral thin wires include two left-handed spiral thin wires disposed at the predetermined proximity gap corresponding to the predetermined proximity interval as one pair of the left-handed spiral thin wire pair, and N pairs of left-handed spiral thin wire pairs as the predetermined adjacent pair gap corresponding to the predetermined adjacent pair interval, performing hand correction shaping by a worker to make the predetermined proximity gap between the right-handed spiral thin wire pair and the left-handed spiral thin wire pair into a minute gap that is five times or less the wire diameter of the thin wire including a contact state, and with a predetermined stitch gap formed by intersecting the right-handed spiral thin wire pair and the left-handed spiral thin wire pair in the plain weave shape as an axial gap of [(predetermined spiral pitch)−{2×(wire diameter of thin wire)}−(minute gap)] and a circumferential gap of [{(stent inner circumferential length corresponding to stent inner diameter)/N}−{2×(wire diameter of thin wire)}−(minute gap)], performing shape memory processing by heating exceeding a transformation point of the thin wire made of a shape memory material in a state where the stent precursor having the predetermined stitch gap is wound around the winding shaft.

According to the above configuration, since the stent precursor production device capable of setting the interval between the adjacent bobbin carriers to different intervals of the predetermined proximity interval and the predetermined adjacent pair interval is used, the gap between the adjacent spiral thin wires can be set to different gaps also for the spiral thin wire formed by winding the thin wire from the bobbin carrier around the winding shaft for the stent precursor. Since the shape memory processing is performed after the predetermined proximity interval is shaped into the minute gap by the hand correction shaping, the gap between the two spiral thin wires constituting one pair of the spiral thin wire pair can be made into the minute gap.

Then, the predetermined stitch gap is formed by intersecting N pairs of the right-handed spiral thin wire pairs and N pairs of the left-handed spiral thin wire pairs in a plain weave shape with two spiral thin wires disposed in the minute gap as one pair. As compared with the case where the predetermined stitch gap is formed by intersecting the N right-handed spiral thin wires and the N left-handed spiral thin wires in a plain weave shape, the number of spiral thin wires per unit area of the outer peripheral surface of the stent is doubled while the predetermined stitch gap is the same, in a manner that the blood vessel wall expansion force can be approximately doubled.

### Advantageous Effects of Invention

According to the stent, the stent precursor production device, and the stent production method configured as described above, without using a special material or a placing structure, the blood vessel wall expansion force is improved while having the stitch gap of the same size under the same other conditions.

### BRIEF DESCRIPTION OF DRAWINGS

FIG. 1 is a side view of a stent according to an embodiment.

FIG. 2 is a flowchart illustrating a procedure of a stent production method according to the embodiment.

FIG. 3 is a perspective view of a stent precursor production device according to the embodiment.

FIG. 4 is a view illustrating a one-side traveling path and an other-side traveling path with only two bobbin carriers with knitting yarn bobbins left on an upper surface of a main body housing portion of the stent precursor production device in FIG. 3.

FIG. 5 is a perspective view of a bobbin carrier with a knitting yarn bobbin.

FIG. 6 is a view illustrating six bobbin carrier pairs disposed on each of the one-side traveling path and the other-side traveling path.

FIG. 7 is a view illustrating six pairs of bobbin carriers disposed on the one-side traveling path extracted from FIG. 6.

FIG. 8 is a view illustrating six pairs of bobbin carriers disposed on the other-side traveling path extracted from FIG. 6.

FIG. 9 is a view illustrating a predetermined proximity interval and a predetermined adjacent pair interval by extracting two adjacent bobbin carrier pairs in the one-side traveling path from FIG. 6.

FIG. 10 is a view illustrating a predetermined proximity interval and a predetermined adjacent pair interval by extracting two adjacent bobbin carrier pairs in the other-side traveling path from FIG. 6.

FIG. 11 is a view illustrating four thin wires pulled out from two pairs of adjacent bobbin carriers in the one-side traveling path with respect to FIG. 9.

FIG. 12 is a view illustrating four thin wires pulled out from two pairs of adjacent bobbin carriers in the other-side traveling path with respect to FIG. 10.

FIG. 13 is a view illustrating a state where four right-handed spiral thin wires formed by the four thin wires in FIG. 9 and four left-handed spiral thin wires formed by the four thin wires in FIG. 12 are knitted by being obliquely intersected in a plain weave shape on a winding shaft.

FIG. 14 is a view illustrating a relationship between a revolution speed of a bobbin carrier driving portion and an axial movement speed of a winding shaft movement mechanism, and a stitch gap.

FIG. 15 is a view illustrating a state where hand correction shaping is partially performed on a hose-shaped stent precursor knitted with 12 right-handed spiral thin wires formed by the six pairs of bobbin carriers on the one-side traveling path and 12 left-handed spiral thin wires formed by the six pairs of bobbin carriers on the other-side traveling path in FIG. 6.

FIG. 16 is a view illustrating, as a comparative example, a stent knitted with six right-handed spiral thin wires in the one-side traveling path and six left-handed spiral thin wires in the other-side traveling path with the same stitch gap as in FIG. 1.

FIG. 17 is a view illustrating, as a comparative example, that the stitch gap is narrowed in a stent knitted with 12 right-handed spiral thin wires in the one-side traveling path and 12 left-handed spiral thin wires in the other-side traveling path.

### DESCRIPTION OF EMBODIMENTS

Hereinafter, embodiments according to the present disclosure will be described in detail with reference to the drawings. Hereinafter, the “superelastic metal” refers to a nickel-titanium alloy called nitinol, an alloy to which copper, cobalt, chromium, iron, or the like is added as necessary, a nickel-aluminum alloy, or various other metals, which has an elastic range 5 to 10 times that of a normal metal by heat treatment and is imparted with superelastic characteristics capable of returning to an original shape even when large deformation is applied.

In the following description, various braid knitting methods are used as the knitting method of the “hose-shaped body”, but any knitting method may be used so long as the metal thin wires can be extended and narrowly converged when a tensile force is applied to the obtained hose-shaped body in the longitudinal direction, and the metal thin wires can be elastically restored to the original hose-shaped body when the tension is released.

The materials and shapes described below are examples and can be appropriately changed in accordance with the specifications of the stent, the stent precursor production device, and the stent production method. In addition, dimensions such as the stitch gap, the wire diameter of the thin wire, and the stent inner diameter, the number of thin wires, the number of carriers, and the like are examples, and can be appropriately changed in accordance with the specification of the stent, particularly, the specification of the “microcatheter that guides the coil” assisted by the stent. Hereinafter, the same reference numerals are given to the same elements in all the drawings, and redundant description will be omitted.

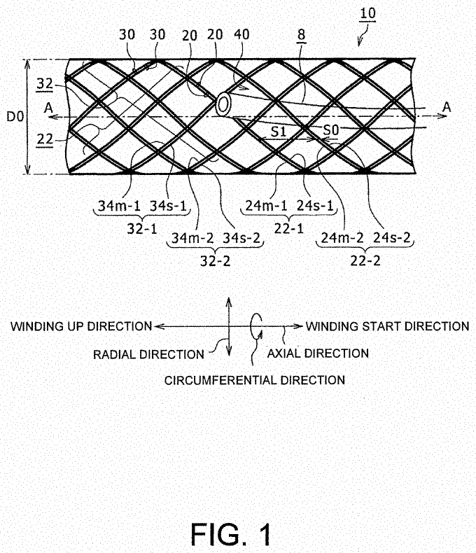

FIG. 1 is a side view of a stent 10. The stent 10 is obtained by knitting thin wires 20 and 30 made of a shape memory alloy; that is, a superelastic metal, as two types of spiral thin wire pairs 22 and 32 of right winding and left winding into a hose-shaped body by braiding the right winding and the left winding into a plain weave shape while obliquely intersecting each other. When the two types of spiral thin wire pairs 22 and 32 are distinguished, they are referred to as a right-handed spiral thin wire pair 22 and a left-handed spiral thin wire pair 32.

The thin wires 20 and 30 are superelastic metal wire materials, nitinol is used as the superelastic metal, and the thin wires 20 and 30 are nitinol thin wires. A wire diameter d0 of the thin wires 20 and 30 is about 30 μm. This is an example, and a superelastic metal thin wire other than the nitinol thin wire can be used, and the wire diameter d0 may be other than about 30 μm, and may be, for example, about 50 μm. Although the thin wires 20 and 30 are the same, the thin wire 20 is distinguished as a nitinol thin wire constituting the right-handed spiral thin wire pair 22, and the thin wire 30 is distinguished as a nitinol thin wire constituting the left-handed spiral thin wire pair 32.

In the stent 10, the right-handed spiral thin wire pair 22 and the left-handed spiral thin wire pair 32 are not knitted in close contact with each other but are knitted while forming a predetermined stitch gap 40. Therefore, when seen from the outer peripheral surface on the front side of the hose-shaped body, the right-handed spiral thin wire pair 22 and the left-handed spiral thin wire pair 32 on the outer peripheral surface on the back side are seen through the predetermined stitch gap 40, but in FIG. 1, only the right-handed spiral thin wire pair 22 and the left-handed spiral thin wire pair 32 on the outer peripheral surface on the front side of the hose-shaped body are illustrated. The right-handed spiral thin wire pair 22 and the left-handed spiral thin wire pair 32 on the outer peripheral surface on the back side are not illustrated.

FIG. 1 illustrates the axial direction, the radial direction, and the circumferential direction of the stent 10. The axial direction is a direction in which a central axis A-A of the hose-shaped body extends and is a direction in which the plurality of right-handed spiral thin wire pairs 22 and the plurality of left-handed spiral thin wire pairs 32 are knitted in a plain weave shape while obliquely intersecting each other. In FIG. 1, the right side of the paper surface is the knitting start direction, and the left side is the knitting progress direction. The radial direction is a radial direction of the hose-shaped body. The circumferential direction is a direction around the central axis A-A of the hose-shaped body.

The right-handed spiral thin wire pair 22 is wound clockwise around the central axis A-A of the hose-shaped body when the hose-shaped body is viewed from the knitting start direction. The left-handed spiral thin wire pair 32 is wound counterclockwise around the central axis A-A. When counted on a cross section perpendicular to the axial direction, N pairs of right-handed spiral thin wire pairs 22 and N pairs of left-handed spiral thin wire pairs 32 are wound around one circumference of the stent 10 in the circumferential direction. Hereinafter, for the stent 10, N=6. Therefore, six pairs of right-handed spiral thin wire pairs 22 and six pairs of left-handed spiral thin wire pairs 32 are set as a repeating unit, and the knitting is repeated from the knitting start direction toward the knitting progress direction along the central axis A-A and knitted to a predetermined stent length. Since N=6, that is a repeating unit of the right-handed spiral thin wire pairs 22 and the left-handed spiral thin wire pairs 32, is used as a repeating unit of each element, N=6 is referred to as a repeating unit N in the following description unless otherwise specified.

The six pairs of right-handed spiral thin wire pairs 22 are distinguished and referred to as right-handed spiral thin wire pairs 22-1, 22-2, 22-3, 22-4, 22-5, and 22-6 in order from the knitting start direction to the knitting progress direction. The six pairs of left-handed spiral thin wire pairs 32 are distinguished and referred to as left-handed spiral thin wire pairs 32-1, 32-2, 32-3, 32-4, 32-5, and 32-6 in order from the knitting start direction to the knitting progress direction. This continues for a predetermined stent length along the axial direction. FIG. 1 illustrates a positional relationship between the right-handed spiral thin wire pairs 22-1 and 22-2 and the left-handed spiral thin wire pairs 32-1 and 32-2 in the vicinity of the knitting start position of the stent 10 knitted to a predetermined stent length.

One right-handed spiral thin wire pair 22 includes two right-handed spiral thin wires 24 disposed with a minute gap S0. When the two right-handed spiral thin wires 24 disposed with the minute gap S0 are distinguished, the right-handed spiral thin wire 24 disposed towards the knitting side along the axial direction is referred to as a right-handed spiral thin wire 24m, and that disposed towards the knitting start side is referred to as a right-handed spiral thin wire 24s. Letters m and s indicate main and sub.

The N pairs of the right-handed spiral thin wire pairs 22 include a total of 2N right-handed spiral thin wires 24. In the 2N right-handed spiral thin wires 24, the gaps along the axial direction of the adjacent right-handed spiral thin wires 24 are not the same.

In the example of FIG. 1, the two pairs of the right-handed spiral thin wire pairs 22-1 and 22-2 include four right-handed spiral thin wires 24m-1, 24s-1, 24m-2, and 24s-2. The right-handed spiral thin wire pair 22-1 includes the right-handed spiral thin wires 24m-1 and 24s-1, and the right-handed spiral thin wire pair 22-2 includes the right-handed spiral thin wires 24m-2 and 24s-2.

The gap along the axial direction of the adjacent right-handed spiral thin wire 24m-1 and the right-handed spiral thin wire 24s-1 constituting one right-handed spiral thin wire pair 22-1 is the minute gap S0. The gap along the axial direction of the adjacent right-handed spiral thin wire 24m-2 and the right-handed spiral thin wire 24s-2 constituting another right-handed spiral thin wire pair 22-2 is also the minute gap S0. The gap between the right-handed spiral thin wire 24m-1 and the right-handed spiral thin wire 24m-2, or the gap between the right-handed spiral thin wire 24s-1 and the right-handed spiral thin wire 24s-2 is a predetermined adjacent pair gap S1 between the right-handed spiral thin wire pairs 22-1 and 22-2 adjacent to each other.

The predetermined adjacent pair gap S1 corresponds to a predetermined spiral pitch along the axial direction of the plurality of right-handed spiral thin wires 24m and a predetermined spiral pitch along the axial direction of the plurality of right-handed spiral thin wires 24s and is set under the operating conditions of a stent precursor production device 50. On the other hand, regarding the minute gap S0, in order to make the predetermined stitch gap 40 in the stent 10 as large as possible, the gap between the two right-handed spiral thin wires 24m and 24s constituting the same pair of the right-handed spiral thin wire pair 22 is set to be as small as possible by, for example, hand correction shaping by the operator. The minute gap S0 may include a contact state and is desirably five times or less the wire diameter of the thin wire even when not in contact. In the above example, since the wire diameter d0 of the thin wire=about 30 μm, for example, the minute gap S0 along the axial direction of the right-handed spiral thin wire 24m and the right-handed spiral thin wire 24s is about 150 μm or less. Since the predetermined adjacent pair gap S1 has a predetermined spiral pitch, the predetermined adjacent pair gap S1 is considerably larger than the minute gap S0.

Using the minute gap S0 and the predetermined adjacent pair gap S1, the gaps between the adjacent right-handed spiral thin wires 24 are arranged in the four right-handed spiral thin wires 24m-1, 24s-1, 24m-2, and 24s-2 constituting the two pairs of the right-handed spiral thin wire pairs 22-1 and 22-2 as follows.

Gap between the right-handed spiral thin wire 24m-1 and the right-handed spiral thin wire 24s-1=S0

Gap between the right-handed spiral thin wire 24s-1 and the right-handed spiral thin wire 24m-2=(S1−S0)

Gap between the right-handed spiral thin wire 24m-2 and the right-handed spiral thin wire 24s-2=S0

Similarly, the two pairs of the left-handed spiral thin wire pairs 32-1 and 32-2 include four left-handed spiral thin wires, a left-handed spiral thin wire 34m-1, a left-handed spiral thin wire 34s-1, a left-handed spiral thin wire 34m-2, a the left-handed spiral thin wire 34s-2. The left-handed spiral thin wire pair 32-1 includes the left-handed spiral thin wires 34m-1 and 34s-1, and the left-handed spiral thin wire pair 32-2 includes left-handed spiral thin wires 34m-2 and 34s-2. As with the case of the right-handed spiral thin wire pair 22, the gap along the axial direction of the left-handed spiral thin wire 34m-1 and the left-handed spiral thin wire 34s-1 constituting one left-handed spiral thin wire pair 32-1 is the minute gap S0. As with the case of the right-handed spiral thin wire pair 22, the gap between the left-handed spiral thin wire 34m-1 and the left-handed spiral thin wire 34m-2, or the gap between the left-handed spiral thin wire 34s-1 and the left-handed spiral thin wire 34s-2 is the predetermined adjacent pair gap S1.

In the stent 10, the predetermined stitch gap 40 formed by knitting the right-handed spiral thin wire pair 22 and the left-handed spiral thin wire pair 32 in an obliquely intersecting manner in a plain weave shape is set to be larger than the outer diameter of a microcatheter 8 used for coil embolization. As a result, the microcatheter 8 for guiding a coil is disposed at a predetermined stent inner diameter D0 of the stent 10, and the microcatheter 8 can be inserted toward the inside of the vascular aneurysm via the predetermined stitch gap 40 in the vicinity of the vascular aneurysm where the coil embolization is performed. This method is referred to as the transcell method, and the stent 10 is a coil-assisted stent 10 that enables the transcell method.

To describe the minute gap S0 and the predetermined adjacent pair gap S1, (S1−S0) is set to a size that allows the microcatheter 8 to pass through. Since the shape of the predetermined stitch gap 40 can be made substantially square by appropriately setting the knitting condition of the hose-shaped body, the diagonal line length of the predetermined stitch gap 40 is about 1.4 a, where the length of each side of the predetermined stitch gap 40 is a, and this corresponds to (S1−S0). In order to pass the microcatheter 8 through the predetermined stitch gap 40, {(S1−S0)/1.4}=a>b must hold, where the outer diameter of the microcatheter 8 is b. This is rewritten as (S1−S0)>(1.4×b).

The unit French (Fr) that is often used for stents and catheters is 1 mm=3 Fr. As the outer diameter of the microcatheter 8 that guides the coil used for coil embolization of a vascular aneurysm, 1.7 Fr=0.56 mm, 2.1 Fr=0.69 mm, and 2.7 Fr=0.89 mm are used. Since a thin outer diameter is suitable for the microcatheter 8 used for coil embolization of a cerebral aneurysm, 1.7 Fr=0.56 mm is used. When b=1.7 Fr=0.56 mm, (1.4×b)=0.78 mm and (S1−S0)>0.78 mm are conditions. From the above, since S0 is about 150 μm=about 0.15 mm at the maximum, S1>0.63 mm, and the predetermined adjacent pair gap S1 is considerably larger than the minute gap S0.

In other words, by narrowing the minute gap S0 as much as possible, the size of the predetermined stitch gap 40 of the stent 10 can be brought considerably close to the stitch gap formed by obliquely intersecting the two right-handed spiral thin wires 24m and the two left-handed spiral thin wires 34m separated by the predetermined adjacent pair gap S1. That is, the size of the predetermined stitch gap 40 of the stent 10 formed by obliquely intersecting the four right-handed spiral thin wires 24 and the four left-handed spiral thin wires 34 becomes close to the stitch gap formed by obliquely intersecting the two right-handed spiral thin wires 24m and the two left-handed spiral thin wires 34m.

As compared with a stent 12 (see FIG. 16) formed by surrounding a stitch gap 42 of the same size with a total of four spiral thin wires 24 and 34 of the two right-handed spiral thin wires 24 and the two left-handed spiral thin wires 34, the blood vessel wall expansion force of the stent 10 disposed in the blood vessel is about two times the blood vessel wall expansion force of the stent 12. That is, in a case where the stents 10 and 12 that are shape memory alloys are placed in the blood vessel, the blood vessel wall expansion force, a force for expanding the blood vessel wall in order to return to the stent outer diameter of which the shape is memorized, is larger as the spiral thin wires 24 and 34 per unit area of the outer peripheral surfaces of the stents 10 and 12 are larger. Since the number of the thin wires 24 and 34 per unit area of the outer peripheral surface of the stent 10 is two times that of the stent 12, the blood vessel wall expansion force of the stent 10 is about two times the blood vessel wall expansion force of the stent 12.

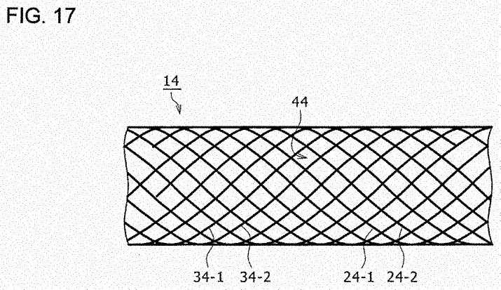

A configuration in which four sides of the predetermined stitch gap 40 of the same size are surrounded by four high-rigidity spiral thin wires using high-rigidity thin wires having rigidity larger than the rigidity of the thin wires 20 and 30 of the stent 10 is conceivable, but if the rigidity of the high-rigidity thin wires is too high, it becomes difficult to knit the high-rigidity thin wire into a hose-shaped body. In addition, in a stent 14 knitted into a hose-shaped body with the number of spiral thin wires 24 and 34 doubled per unit area of the outer peripheral surface while maintaining the wire diameter d0 of the thin wires 20 and 30 at about 30 μm, a stitch gap 44 has a size of about (¼) of the predetermined stitch gap 40 of the stent 10 (see FIG. 17). Therefore, the blood vessel wall expansion force is about two times that of the stent 12 similarly to the stent 10, but the microcatheter 8 cannot be passed through the stitch gap 44.

The stent 10 can secure the size of the predetermined stitch gap 40 through which the microcatheter 8 can pass while doubling the blood vessel wall expansion force. As a result, since the stent 10 has a large blood vessel wall expansion force even if it is placed in the blood vessel, it can be placed at a site of the blood vessel where the vascular aneurysm is present without being swept away by the blood flow. Then, the microcatheter 8 for guiding a coil used for coil embolization is disposed within the predetermined stent inner diameter D0, and the microcatheter 8 can be inserted from the predetermined stitch gap 40 toward the vascular aneurysm.

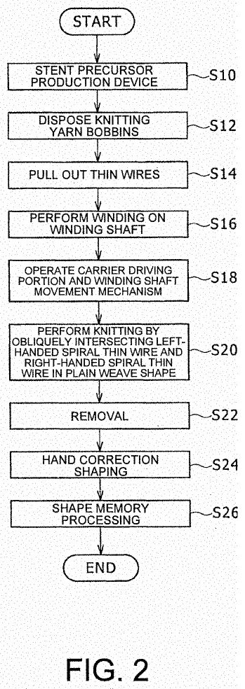

Next, a production method of the stent 10 will be described. FIG. 2 is a flowchart illustrating each procedure of a stent production method. From S10 to S24, the hose-shaped body is knitted in a room temperature atmosphere, and then the shape memory processing at a predetermined temperature in S26 is performed to obtain the stent 10. In this sense, the steps up to S26 before the shape memory processing is performed are steps for obtaining a stent precursor 10Z that is a precursor of the stent 10 subjected to the shape memory processing.

First, the stent precursor production device 50 having a predetermined specification is prepared (S10). The stent precursor production device 50 has a specification of the repeating unit N=6 and has a structure of a braid knitting machine in which 2N=12 right-handed spiral thin wires 24 and 2N=12 left-handed spiral thin wires 34 are obliquely intersected with each other and knitted in a plain weave shape. The specification different from the general braid knitting machine includes that different gaps such as a predetermined proximity gap S2 and the predetermined adjacent pair gap S1 wider than the predetermined proximity gap S2 can be set for each of the gap between the two adjacent right-handed spiral thin wires 24 and the gap between the two adjacent left-handed spiral thin wires 34.

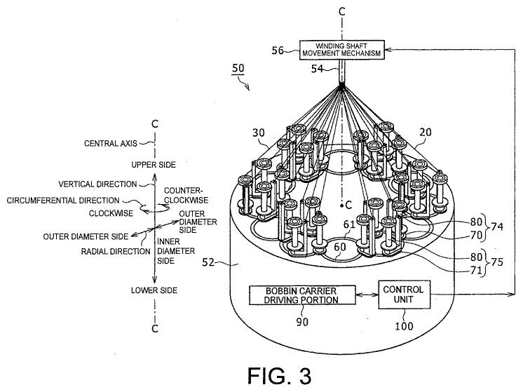

FIG. 3 illustrates a configuration of the stent precursor production device 50. The stent precursor production device 50 includes a main body housing portion 52 having a cylindrical outer shape and serving as a base of the entire device, and a winding shaft movement mechanism 56 that is disposed above the main body housing portion 52 and moves and drives a winding shaft 54 in the axial direction.

FIG. 3 illustrates a vertical direction, a radial direction, and a circumferential direction in the stent precursor production device 50. The vertical direction is a direction parallel to a central axis C-C passing through a cylindrical center C of the main body housing portion 52, and a direction in which the winding shaft 54 is disposed is an upper side and the opposite side is a lower side. The radial direction is a radiating direction from the central axis C-C; a direction toward the central axis C-C is an inner diameter side, and a direction away from the central axis C-C is an outer diameter side. The circumferential direction is a direction around the central axis C-C. When viewed from the upper side to the lower side of the central axis C-C, the direction around the right screw is clockwise, and the direction around the left screw is counterclockwise.

The winding shaft 54 is disposed on the central axis C-C of the main body housing portion 52. The winding shaft 54 is a cylindrical rod having a predetermined outer diameter corresponding to the predetermined stent inner diameter D0. In this case, the predetermined outer diameter is set to D0. The winding shaft 54 corresponds to a shape constraining jig at the time of heat treatment for imparting superelasticity to the stent precursor 10Z.

The winding shaft movement mechanism 56 is a movement mechanism that detachably holds the winding shaft 54 and moves the winding shaft 54 at an axial movement speed along the axial direction of the central axis C-C.

Two traveling paths 60 and 61 provided on the upper surface of the main body housing portion 52 are groove paths in which N=6 annular grooves 62 and N=6 annular grooves 63 are continuously connected while being alternately disposed and are disposed to make one round in a circumferential shape as a whole. The distinction between the traveling paths 60 and 61 and the relationship between the traveling paths 60 and 61 and the annular grooves 62 and 63 will be described later.

A knitting yarn bobbin 80 is a cylindrical thin wire bobbin around which the thin wires 20 and 30 made of a shape memory material are wound in advance. The thin wire 20 or the thin wire 30 is only distinguished by reference signs depending on whether the thin wire is wound around the winding shaft 54 to become the right-handed spiral thin wire 24 or the left-handed spiral thin wire 34, and there is no distinction in a state of being wound around the knitting yarn bobbin 80 with the same material, wire diameter, and the like.

Bobbin carriers 70 and 71 are provided integrally with the knitting yarn bobbin 80 and are for transporting the knitting yarn bobbin 80 along the traveling paths 60 and 61. Hereinafter, the bobbin carrier 70 is referred to as a carrier 70, and the bobbin carrier 71 is referred to as a carrier 71 unless otherwise specified.

The traveling path 60 is a groove path through which the carrier 70 travels clockwise along the circumferential direction of the main body housing portion 52, and the traveling path 61 is a groove path through which the carrier 71 travels counterclockwise along the circumferential direction of the main body housing portion 52. Although the basic structures of the carriers 70 and 71 are the same, a carrier disposed on the traveling path 60 and traveling clockwise along the circumferential direction of the main body housing portion 52 is referred to as the carrier 70, and a carrier disposed on the traveling path 61 and traveling counterclockwise along the circumferential direction of the main body housing portion 52 is referred to as the carrier 71. In other words, the carrier 70 is for transporting the knitting yarn bobbin 80 along the traveling path 60, and the carrier 71 is for transporting the knitting yarn bobbin 80 along the traveling path 61.

The carrier 70 on which the knitting yarn bobbin 80 is mounted is referred to as a bobbin-attached carrier 74 to be distinguished from the carrier 70 on which the knitting yarn bobbin 80 is not mounted, and the carrier 71 on which the knitting yarn bobbin 80 is mounted is referred to as a bobbin-attached carrier 75 to be distinguished from the carrier 71 on which the knitting yarn bobbin 80 is not mounted. 2N=12 bobbin-attached carriers 74 are disposed on the traveling path 60, and 2N=12 bobbin-attached carriers 75 are disposed on the traveling path 61. In FIG. 3, two of 12 bobbin-attached carriers 74 and 75 are denoted by reference signs, and the bobbin-attached carrier 74 in which the knitting yarn bobbin 80 is mounted on the carrier 70 and the bobbin-attached carrier 75 in which the knitting yarn bobbin 80 is mounted on the carrier 71 are illustrated.

A bobbin carrier driving portion 90 is a driving device that drives a total of 4N=24 knitting yarn bobbins 80 at a predetermined revolution speed around the revolution axis with the central axis C-C as the revolution axis aligning 2N=12 bobbin-attached carriers 74 along the traveling path 60 and aligning 2N=12 bobbin-attached carriers 75 along the traveling path 61.

A control unit 100 is a control device connected to the winding shaft movement mechanism 56 and the bobbin carrier driving portion 90 by an appropriate signal line and controls the axial movement speed of the winding shaft movement mechanism 56 and the revolution speed of the bobbin carrier driving portion 90.

FIG. 3 illustrates a state where the knitting yarn bobbin 80 is already disposed on the carrier 70, and the thin wires 20 and 30 are pulled out from the knitting yarn bobbin 80 and wound around the winding shaft 54 in the stent precursor production device 50. The state of the stent precursor production device 50 in the initial state is a state where only 2N=12 carriers 70 are disposed on the traveling path 60 and 2N=12 carriers 71 are disposed on the traveling path 61. From such a state, one knitting yarn bobbin 80 is disposed in each of the 2N=12 carriers 70 and the 2N=12 carriers 71; that is, 4N=24 in total are disposed (S12).

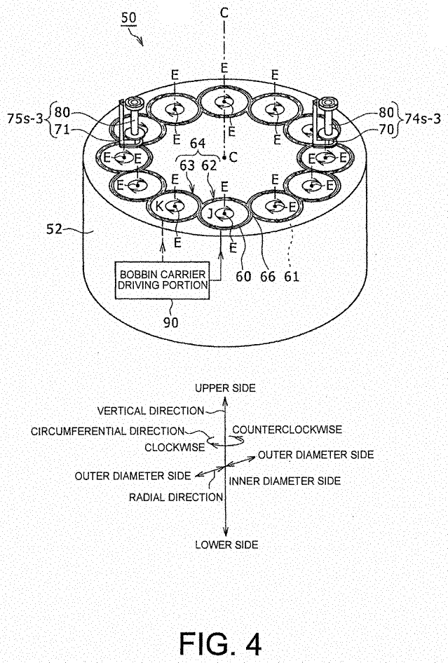

FIG. 4 is a view illustrating a state where one of the 2N=12 bobbin-attached carriers 74 and one of the 2N=12 bobbin-attached carriers 75 are left in a manner that the traveling paths 60 and 61 on the upper surface of the main body housing portion 52 of the stent precursor production device 50 in FIG. 3 appear. Formation of the traveling path 60 on which the carrier 70 travels clockwise along the circumferential direction of the main body housing portion 52 and the traveling path 61 on which the carrier 71 travels counterclockwise along the circumferential direction of the main body housing portion 52 will be described with reference to FIG. 4. In the following drawings, the traveling path 60 is indicated by a solid line, and the traveling path 61 is indicated by a broken line.

The two traveling paths 60 and 61 are groove paths in which N=6 annular grooves 62 and N=6 annular grooves 63 are continuously connected around the central axis C-C of the main body housing portion 52 while being alternately disposed and are disposed to make one round in a circumferential shape as a whole. A driving mechanism (not illustrated) that engages with the driving ends of the carriers 70 and 71 to drive the carriers 70 and 71 in a manner that the carriers 70 and 71 travel along the traveling paths 60 and 61 is disposed in the main body housing portion 52 on the lower side of the annular grooves 62 and 63. The driving mechanism is driven by the bobbin carrier driving portion 90 under the control of the control unit 100. The carriers 70 and 71 having the driving ends inserted into the annular grooves 62 and 63 travel along the annular grooves 62 and 63.

A difference between the annular groove 62 and the annular groove 63 is a direction in which the carriers 70 and 71 travel around a central axis E-E passing through a center E of the annular grooves 62 and 63. When the bobbin carrier driving portion 90 is operated, the annular groove 62 causes the carriers 70 and 71 to travel clockwise around the central axis E-E, and the annular groove 63 causes the carriers 70 and 71 to travel counterclockwise around the central axis E-E by the driving mechanism. Therefore, the annular groove 62 is referred to as a clockwise annular groove 62, the annular groove 63 is referred to as a counterclockwise annular groove 63, and a position where the clockwise annular groove 62 and the counterclockwise annular groove 63 are connected is referred to as an intersection position 66, since the grooves intersect each other.

In one clockwise annular groove 62, since the counterclockwise annular groove 63 is disposed on both sides, there are intersection positions 66 on both sides. The clockwise annular groove 62 will be described separately as an inner diameter side groove portion on the inner diameter side and an outer diameter side groove portion on the outer diameter side along the radial direction of the main body housing portion 52 between the intersection positions 66 on both sides. The carrier 70 traveling clockwise along the circumferential direction of the main body housing portion 52 travels in the outer diameter side groove portion of the clockwise annular groove 62. On the other hand, the carrier 71 traveling counterclockwise along the circumferential direction of the main body housing portion 52 travels in the inner diameter side groove portion of the clockwise annular groove 62.

In one counterclockwise annular groove 63, since the clockwise annular groove 62 is disposed on both sides, there are intersection positions 66 on both sides. The counterclockwise annular groove 63 will be described separately as an inner diameter side groove portion on the inner diameter side and an outer diameter side groove portion on the outer diameter side along the radial direction of the main body housing portion 52 between the intersection positions 66 on both sides. The carrier 71 traveling counterclockwise along the circumferential direction of the main body housing portion 52 travels in the outer diameter side groove portion of the counterclockwise annular groove 63. On the other hand, the carrier 70 traveling clockwise along the circumferential direction of the main body housing portion 52 travels in the inner diameter side groove portion of the counterclockwise annular groove 63.

In FIG. 4, one clockwise annular groove 62 is denoted by J, and with respect to J, the counterclockwise annular groove 63 connected at the intersection position 66 of the clockwise end along the circumferential direction of the main body housing portion 52 is denoted by K.

The carrier 70 having traveled in the outer diameter side groove portion indicated by a solid line of the clockwise annular groove 62 denoted by J switches the traveling path to the inner diameter side groove portion indicated by a solid line of the counterclockwise annular groove 63 denoted by K at the intersection position 66. As a result, the carrier 70 moves from the outer diameter side groove portion of the clockwise annular groove 62 denoted by J that has been traveled so far to the inner diameter side groove portion of the counterclockwise annular groove 63 denoted by K and continues traveling clockwise along the circumferential direction of the main body housing portion 52.

On the other hand, the carrier 71 having traveled in the outer diameter side groove portion indicated by a broken line of the counterclockwise annular groove 63 denoted by K switches the traveling path to the inner diameter side groove portion of the clockwise annular groove 62 denoted by J at the intersection position 66. As a result, the carrier 71 moves from the outer diameter side groove portion of the counterclockwise annular groove 63 denoted by K that has been traveled so far to the inner diameter side groove portion of the clockwise annular groove 62 denoted by J and continues traveling counterclockwise along the circumferential direction of the main body housing portion 52.

That is, at the intersection position 66, the distribution of the traveling direction is performed in accordance with whether the carriers 70 and 71 have traveled in the outer diameter side groove portion or the inner diameter side groove portion. The distribution of the traveling direction of the carrier 70 is automatically performed by a carrier distribution mechanism (not illustrated) provided at the intersection position 66. By the action of the carrier distribution mechanism, the carrier 70 can continue to travel clockwise, and the carrier 71 can continue to travel counterclockwise.

As described above, the traveling path 60 is formed by connecting the outer shape portion of the clockwise annular groove 62 denoted by J and the inner diameter groove portion of the counterclockwise annular groove 63 denoted by K at the intersection position 66. In addition, the traveling path 61 is formed by connecting the outer shape portion of the counterclockwise annular groove 63 denoted by K and the inner diameter groove portion of the clockwise annular groove 62 denoted by J at the intersection position 66. When a pair of the clockwise annular groove 62 and the counterclockwise annular groove 63 connected to each other at the intersection position 66 is referred to as an annular groove pair 64, the traveling path 60 and the traveling path 61 intersect each other at an intersection position 66 of one annular groove pair 64 in substantially a figure-eight shape.

The traveling paths 60 and 61 on the upper surface of the main body housing portion 52 of the stent precursor production device 50 are groove paths in which the annular groove pair 64 is continuously connected around the central axis C-C of the main body housing portion 52 with the repeating unit N=6 and disposed to make one round in a circumferential shape as a whole. As illustrated in FIG. 4, the traveling paths 60 and 61 are a pair of meandering paths meandering around the central axis C-C of the main body housing portion 52 that intersect each other at the intersection position 66 of 2N=12. Hereinafter, the pair of meandering paths are distinguished, and the traveling path 60 is referred to as a one-side traveling path 60, and the traveling path 61 is referred to as an other-side traveling path 61.

In FIG. 4, one of the two illustrated bobbin-attached carriers 74 and 75 is a bobbin-attached carrier 74s-3 disposed on the one-side traveling path 60 and having the knitting yarn bobbin 80 mounted on the carrier 70. Another is a bobbin-attached carrier 75s-3 disposed on the other-side traveling path 61 and having the knitting yarn bobbin 80 mounted on the carrier 71. The reference signs of 74s-3 and 75s-3 are reference signs used to distinguish the bobbin-attached carriers 74 and the bobbin-attached carriers 75 each having 2N=12 from other bobbin-attached carriers 74 and 75, and details will be described later.

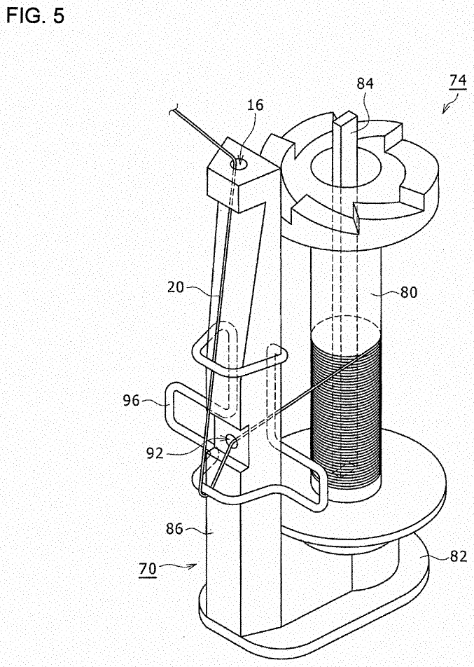

Although there is a difference as to whether the bobbin-attached carriers 74 and 75 are disposed on the one-side traveling path 60 or the other-side traveling path 61, since they are structurally completely the same, the bobbin-attached carrier 74 will be described below. FIG. 5 is a perspective view of the bobbin-attached carrier 74 in which the knitting yarn bobbin 80 is mounted on the carrier 70. A driving end that engages with the driving mechanism is provided on the lower side of the carrier 70, but in FIG. 5, only a portion of the carrier 70 on the upper side of the upper surface of the main body housing portion 52 of the stent precursor production device 50 is illustrated, and the driving end and the like are not illustrated.

In the bobbin-attached carrier 74, the knitting yarn bobbin 80 is a cylindrical thin wire bobbin around which the thin wire 20 made of a shape memory material is wound in advance. The carrier 70 is a member in which a bobbin shaft 84 and a yarn passing portion 86 are erected on a bobbin base portion 82. The bobbin shaft 84 is a shaft body that rotatably supports the knitting yarn bobbin 80. The yarn passing portion 86 is a thin wire guiding member that extends the thin wire 20 pulled out from the knitting yarn bobbin 80 upward through a thin wire lead-out hole 92 to guide the thin wire 20 to a thin wire supply hole 94 at a predetermined height position at the upper end. A tension weight 96 is a weight member for applying an appropriate tension to the thin wire 20 passing through the thin wire lead-out hole 92.

Returning to FIG. 2 again, when the knitting yarn bobbin 80 is disposed on the carrier 70 in S12, the thin line wound around the knitting yarn bobbin 80 is unwound and pulled out from the thin wire supply hole 94 provided at the upper end of the yarn passing portion 86 via the thin wire lead-out hole 92 and the tension weight 96 (S14). A distal end of the thin wire 20 pulled out from the thin wire supply hole 94 is wound around the winding shaft 54. Similarly, in the bobbin-attached carrier 75, the distal end of the thin wire 30 pulled out from the thin wire supply hole 94 is wound around the winding shaft 54 (S16). FIG. 3 illustrates a state where 12 thin wires 20 pulled out from the 12 bobbin-attached carriers 74 and 12 thin wires 30 pulled out from the 12 bobbin-attached carriers 75 are wound around the winding shaft 54.

After S16, the bobbin carrier driving portion 90 and the winding shaft movement mechanism 56 are operated under the control of the control unit 100 (S18). By the operation of the bobbin carrier driving portion 90, with the central axis C-C of the main body housing portion 52 as the revolution axis, the bobbin-attached carrier 74 travels clockwise around the revolution axis on the one-side traveling path 60, and the bobbin-attached carrier 75 travels counterclockwise around the revolution axis on the other-side traveling path 61.

2N=12 bobbin-attached carriers 74 are disposed on the one-side traveling path 60, and 2N=12 bobbin-attached carriers 75 are disposed on the other-side traveling path 61. The 12 bobbin-attached carriers 74 are not disposed at equal intervals on the one-side traveling path 60; two bobbin-attached carriers 74 are set as one bobbin carrier pair 72, and the bobbin carrier pair 72 of N=6 is disposed at equal intervals at a predetermined adjacent pair interval with the bobbin carrier pair 72 as a unit. The interval between the two bobbin-attached carriers 74 in one bobbin carrier pair 72 is disposed at a predetermined proximity interval narrower than the predetermined adjacent pair interval. Similarly, the 12 bobbin-attached carriers 75 are not disposed at equal intervals on the other-side traveling path 61; two bobbin-attached carriers 75 are set as one bobbin carrier pair 73, and the bobbin carrier pair 73 is disposed at equal intervals at a predetermined adjacent pair interval with the bobbin carrier pair 73 as a unit. The interval between the two bobbin-attached carriers 75 in one bobbin carrier pair 73 is disposed at a predetermined proximity interval narrower than the predetermined adjacent pair interval. Hereinafter, the bobbin carrier pair 72 is referred to as a carrier pair 72, and the bobbin carrier pair 73 is referred to as a carrier pair 73 unless otherwise specified.

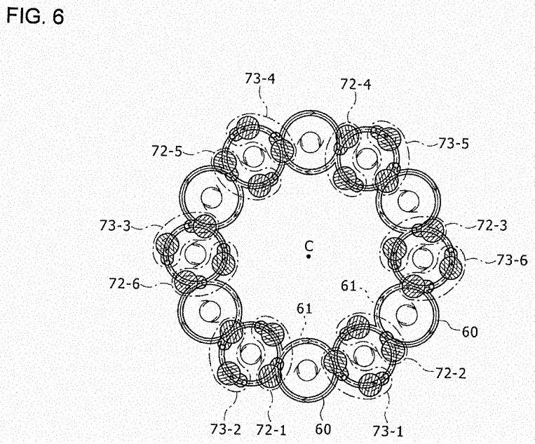

FIG. 6 is a top view of FIG. 3 as viewed from above, illustrating a disposition of the carrier pair 72 of N=6 and the carrier pair 73 of N=6. In FIG. 6, illustration of the winding shaft 54 is omitted, and the cylindrical center C of the main body housing portion 52 is illustrated. Since there are six pairs of carriers 72, these pairs are distinguished and referred to as carrier pairs 72-1, 72-2, 72-3, 72-4, 72-5, and 72-6 in the order of counterclockwise position in a manner that the head side of the clockwise direction with respect to the revolution axis has a smaller number. Similarly, since there are six pairs of carrier pairs 73, these pairs are distinguished and referred to as carrier pairs 73-1, 73-2, 73-3, 73-4, 73-5, and 73-6 in the order of clockwise position in a manner that the head side of the counterclockwise direction with respect to the revolution axis has a smaller number. The head carrier pair 72-1 and the head carrier pair 73-1 may be arbitrarily set, but in FIG. 6 and subsequent figures, the head carrier pair 72-1 and the head carrier pair 73-1 are disposed adjacent to each other on the traveling paths 60 and 61.

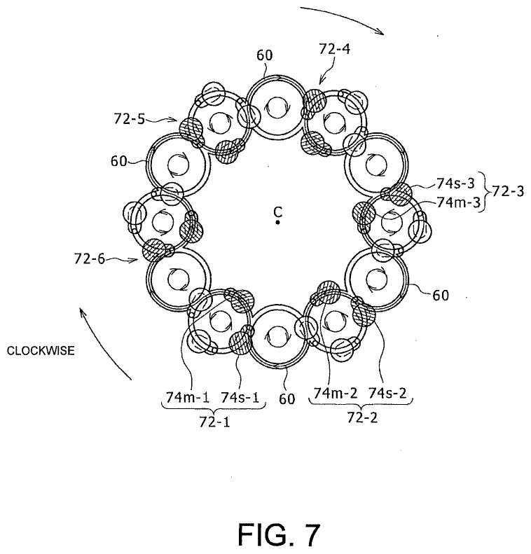

FIG. 7 is a diagram extracted from FIG. 6 by hatching only six pairs of carrier pairs 72. The two bobbin-attached carriers 74 constituting the carrier pair 72 are distinguished, and the bobbin-attached carrier 74 on the head side of the clockwise direction on the other-side traveling path 61 is denoted by a reference sign m, and then the bobbin-attached carrier 74 continuing at a predetermined proximity interval is denoted by a reference sign s.

In the example of FIG. 7, in the carrier pair 72-1, a bobbin-attached carrier 74-1 on the head side of the clockwise direction of the one-side traveling path 60 is referred to as a bobbin-attached carrier 74m-1, and the bobbin-attached carrier 74 continuing from the bobbin-attached carrier 74m-1 at a predetermined proximity interval is referred to as a bobbin-attached carrier 74s-1. Similarly, the two bobbin-attached carriers 74 constituting the carrier pair 72-2 are also referred to as a bobbin-attached carrier 74m-2 and a bobbin-attached carrier 74s-2. In addition, the two bobbin-attached carriers 74 constituting the carrier pair 72-3 are also referred to as a bobbin-attached carrier 74m-3 and the bobbin-attached carrier 74s-3. Hereinafter, the same applies to the carrier pairs 72-4, 72-5, and 72-6. These carrier pairs 72 travel clockwise on the one-side traveling path 60 around the revolution axis at the revolution speed as indicated by arrows in FIG. 7 while maintaining a predetermined proximity interval and a predetermined adjacent pair interval.

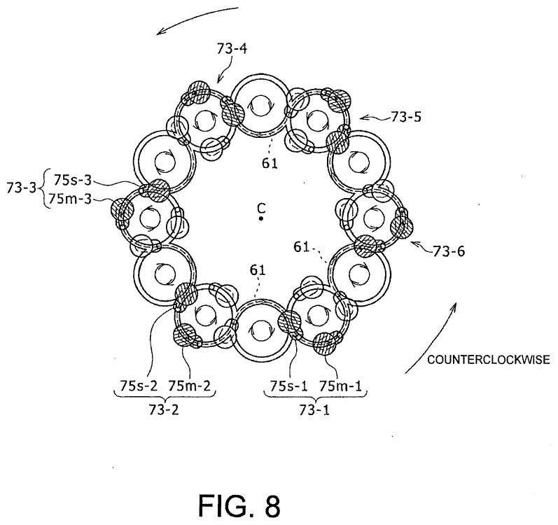

FIG. 8 is a diagram extracted from FIG. 6 by hatching only six pairs of carrier pairs 73. The two bobbin-attached carriers 75 constituting the carrier pair 73 are distinguished, and the bobbin-attached carrier 75 on the head side of the counterclockwise direction on the other-side traveling path 61 is denoted by a reference sign m, and then the bobbin-attached carrier 75 continuing at a predetermined proximity interval is denoted by a reference sign s.

In the example of FIG. 8, in the carrier pair 73-1, a bobbin-attached carrier 75-1 on the head side of the counterclockwise direction of the other-side traveling path 61 is referred to as a bobbin-attached carrier 75m-1, and the bobbin-attached carrier 75 continuing from the bobbin-attached carrier 75m-1 at a predetermined proximity interval is referred to as a bobbin-attached carrier 75s-1. Similarly, the two bobbin-attached carriers 75 constituting the carrier pair 73-2 are also referred to as a bobbin-attached carrier 75m-2 and a bobbin-attached carrier 75s-2. In addition, the two bobbin-attached carriers 75 constituting the carrier pair 73-3 are also referred to as a bobbin-attached carrier 75m-3 and a bobbin-attached carrier 75s-3. Hereinafter, the same applies to the carrier pairs 73-4, 73-5, and 73-6. These carrier pairs 73 travel counterclockwise on the other-side traveling path 61 around the revolution axis at the revolution speed as indicated by arrows in FIG. 8 while maintaining a predetermined proximity interval and a predetermined adjacent pair interval.

In FIG. 4, the bobbin-attached carrier 74s-3 is illustrated with the knitting yarn bobbin 80 disposed on the carrier 70, and the bobbin-attached carrier 75s-3 is illustrated with the knitting yarn bobbin 80 disposed on the carrier 71.

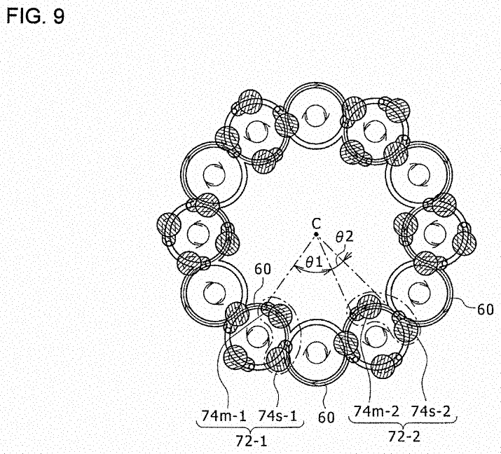

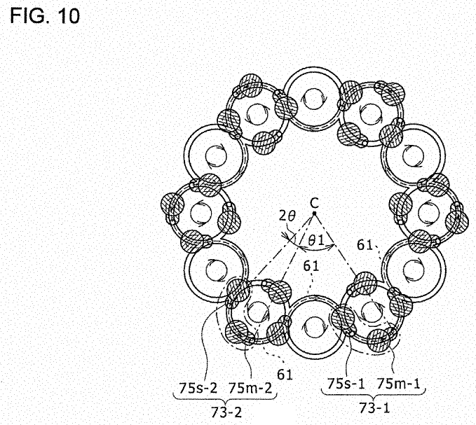

FIGS. 9 and 10 are views illustrating a predetermined proximity interval θ2 and a predetermined adjacent pair interval θ1. FIG. 9 is a view illustrating the predetermined proximity interval θ2 and the predetermined adjacent pair interval θ1 at a visual angle around a revolution axis C when the carrier pair 72-1 and the carrier pair 72-2 are extracted as the adjacent carrier pairs 72 from FIG. 6. Similarly, FIG. 10 is a view illustrating the predetermined proximity interval θ2 and the predetermined adjacent pair interval θ1 at a visual angle around a revolution axis C when the carrier pair 73-1 and the carrier pair 73-2 are extracted as the adjacent carrier pairs 73 from FIG. 6.

In the carrier pair 72 and the carrier pair 73, the predetermined adjacent pair interval θ2 is an angular interval of the same size. Since six pairs of carrier pairs 72 are disposed around the revolution axis C and six pairs of carrier pairs 73 are also disposed around the revolution axis C, the predetermined adjacent pair interval θ1 is an angular interval of 60 degrees.

In the carrier pair 72 and the carrier pair 73, the predetermined proximity interval θ2 is an angular interval of the same size. The predetermined proximity interval θ2 is preferably set at as small an angular interval as possible, but when the predetermined proximity interval θ2 is set at an angular interval that is too small, traveling interference between two adjacent bobbin-attached carriers 74 or traveling interference between two adjacent bobbin-attached carriers 75 is likely to occur. Therefore, in consideration of the sizes of the bobbin-attached carrier 74 and the bobbin-attached carrier 75, the predetermined proximity interval θ2 is set within a range in which the traveling interference does not occur. In the examples of FIGS. 9 and 10, the predetermined proximity interval θ2 is set to an angular interval of about 15 degrees, which is considerably smaller than the predetermined adjacent pair interval θ1. This is an example, and an angular interval other than 15 degrees can be set in accordance with the specifications of the size of the bobbin-attached carrier 74 and the bobbin-attached carrier 75.

As described in S16, the thin wire 20 is pulled out from each of the 12 bobbin-attached carriers 74, the thin wire 30 is pulled out from each of the 12 bobbin-attached carriers 75, and the distal ends of the thin wires 20 and 30 are wound around the winding shaft 54. Here, when the bobbin carrier driving portion 90 is operated, the bobbin-attached carrier 74 travels clockwise, and the bobbin-attached carrier 75 travels counterclockwise around the revolution axis, in a manner that the thin wires 20 and 30 wound around the knitting yarn bobbin 80 are unwound accordingly. The yarn passing portion 86 is pulled out from the thin wire supply hole 94 and wound along the outer peripheral surface of the winding shaft 54. Here, when the winding shaft movement mechanism 56 is operated and the winding shaft 54 moves upward along the axial direction of the central axis C-C, the thin wire 20 supplied from the bobbin-attached carrier 74 is wound around the outer peripheral surface of the winding shaft 54 at a predetermined spiral pitch in a right-handed spiral shape. Similarly, the thin wire 30 supplied from the bobbin-attached carrier 75 is wound around the outer peripheral surface of the winding shaft 54 at a predetermined spiral pitch in a left-handed spiral shape.

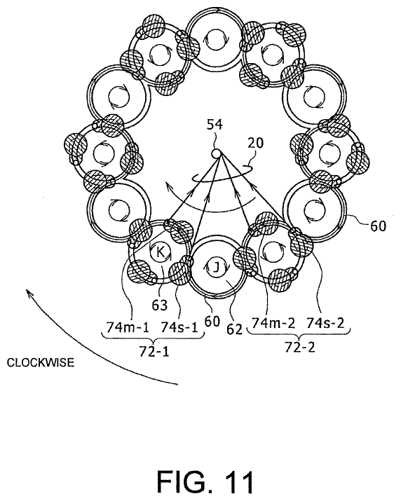

FIG. 11 is a view illustrating four thin wires 20 from the bobbin-attached carriers 74m-1, 74s-1, 74m-2, and 74s-2 constituting the two adjacent carrier pairs 72-1 and 72-2 in FIG. 9. The four thin wires 20 are wound around the outer peripheral surface of the winding shaft 54 in a right-handed spiral shape at a predetermined spiral pitch as the four bobbin-attached carriers 74 travel clockwise on the one-side traveling path 60.

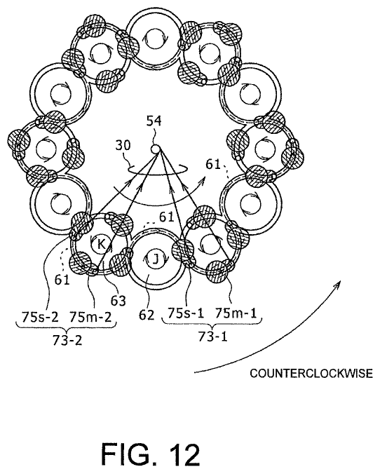

FIG. 12 is a view illustrating four thin wires 30 from the bobbin-attached carriers 75m-1, 75s-1, 75m-2, and 75s-2 constituting the two adjacent carrier pairs 73-1 and 73-2 in FIG. 10. The four thin wires 30 are wound around the outer peripheral surface of the winding shaft 54 in a left-handed spiral shape at a predetermined spiral pitch as the four bobbin-attached carriers 75 travel counterclockwise on the other-side traveling path 61.

The thin wires 20 wound in a right-handed spiral shape and the thin wires 30 wound in a left-handed spiral shape are knitted as the right-handed spiral thin wire 24 and the left-handed spiral thin wire 34 on the outer peripheral surface of the winding shaft 54 in a plain weave shape while obliquely intersecting each other (S20). The method of oblique intersection will be described with reference to FIGS. 11 and 12.

When the carrier pair 72 continues to travel clockwise from the state in FIG. 11 and the carrier pair 73 continues to travel counterclockwise from the state in FIG. 12, the thin wire 20 pulled out from the bobbin-attached carrier 74m-2 and the thin wire 30 pulled out from the bobbin-attached carrier 75m-2 first intersect. In the clockwise annular groove 62 denoted by J in FIGS. 11 and 12, the intersecting occurs when the bobbin-attached carrier 74m-2 comes to the outer diameter side groove portion that is the one-side traveling path 60 and the bobbin-attached carrier 75m-2 comes to the inner diameter side groove portion that is the other-side traveling path 61.

Here, since the thin wire 20 pulled out from the thin wire supply hole 94 of the bobbin-attached carrier 74m-2 is wound around the outer peripheral surface of the winding shaft 54 in a right-handed spiral shape, the thin wire 20 wound around the winding shaft 54 is referred to as the right-handed spiral thin wire 24m-2. Similarly, since the thin wire 30 pulled out from the thin wire supply hole 94 of the bobbin-attached carrier 75m-2 is wound around the outer peripheral surface of the winding shaft 54 in a left-handed spiral shape, the thin wire 30 wound around the winding shaft 54 is referred to as the left-handed spiral thin wire 34m-2. In the intersecting state, since the thin wire supply hole 94 of the bobbin-attached carrier 74m-2 is located on the outer diameter side of the thin wire supply hole 94 of the bobbin-attached carrier 75m-2, on the outer peripheral surface of the winding shaft 54, the right-handed spiral thin wire 24m-2 obliquely intersects in a state of being disposed on the upper side of the left-handed spiral thin wire 34m-2.

Further, when the carrier pair 72 continues to travel clockwise and the carrier pair 73 continues to travel counterclockwise, the thin wire 20 pulled out from the bobbin-attached carrier 74m-2 then intersects the thin wire 30 pulled out from the bobbin-attached carrier 75s-2. In the counterclockwise annular groove 63 denoted by Kin FIGS. 11 and 12, the intersecting occurs when the bobbin-attached carrier 75s-2 comes to the outer diameter side groove portion that is the other-side traveling path 61 and the bobbin-attached carrier 74m-2 comes to the inner diameter side groove portion that is the one-side traveling path 60.

Here, since the thin wire 30 pulled out from the thin wire supply hole 94 of the bobbin-attached carrier 75s-2 is wound around the outer peripheral surface of the winding shaft 54 in a left-handed spiral shape, the thin wire 30 wound around the winding shaft 54 is referred to as the left-handed spiral thin wire 34s-2. In the intersecting state, since the thin wire supply hole 94 of the bobbin-attached carrier 74m-2 is located on the inner diameter side of the thin wire supply hole 94 of the bobbin-attached carrier 75s-2, on the outer peripheral surface of the winding shaft 54, the right-handed spiral thin wire 24m-2 obliquely intersects in a state of being disposed on the lower side of the left-handed spiral thin wire 34s-2.

As described above, on the outer peripheral surface of the winding shaft 54, the right-handed spiral thin wire 24m-2 is disposed above the left-handed spiral thin wire 34m-2 and obliquely intersects, and next, the right-handed spiral thin wire 24m-2 is disposed below the left-handed spiral thin wire 34s-2 and obliquely intersects, and this oblique intersection is sequentially repeated. Therefore, on the outer peripheral surface of the winding shaft 54, with respect to one right-handed spiral thin wire 24, the plurality of left-handed spiral thin wires 34 are knitted in a plain weave shape in which an oblique intersection disposed above the left-handed spiral thin wire 34 and an oblique intersection disposed below the left-handed spiral thin wire 34 are alternately repeated. On the other hand, on the outer peripheral surface of the winding shaft 54, with respect to one left-handed spiral thin wire 34, the plurality of right-handed spiral thin wires 24 are knitted in a plain weave shape in which an oblique intersection disposed above the right-handed spiral thin wire 24 and an oblique intersection disposed below the right-handed spiral thin wire 24 are alternately repeated.

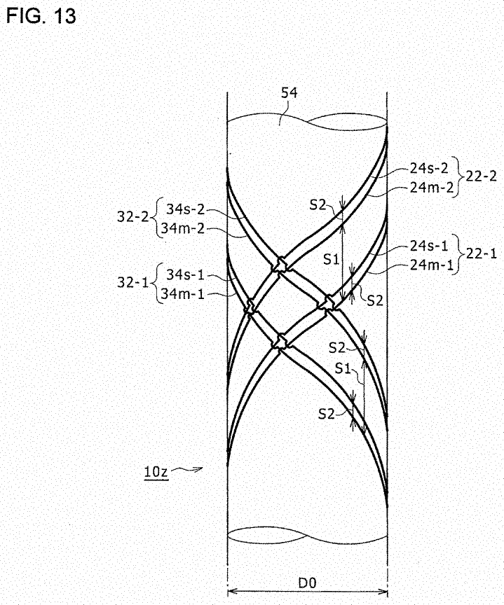

FIG. 13 is a view illustrating an example of the stent precursor 10Z on the outer peripheral surface of the winding shaft 54. FIG. 13 shows the right-handed spiral thin wires 24m-1, 24s-1, 24m-2, and 24s-2 in a state where the thin wires 20 pulled out from the four bobbin-attached carriers 74m-1, 74s-1, 74m-2, and 74s-2 described in FIG. 11 are wound around the outer peripheral surface of the winding shaft 54. Further, FIG. 13 shows the left-handed spiral thin wires 34m-1, 34s-1, 34m-2, and 34s-2 in a state where the thin wires 30 pulled out from the four bobbin-attached carriers 75m-1, 75s-1, 75m-2, and 75s-2 described in FIG. 12 are wound around the outer peripheral surface of the winding shaft 54.

In FIG. 13, a going-over state and a going-through state are illustrated in order to emphasize the oblique intersection. The right-handed spiral thin wire 24m-2 goes through the left-handed spiral thin wire 34s-2, then goes over the left-handed spiral thin wire 34m-2, then goes through the left-handed spiral thin wire 34s-1, and then goes over the left-handed spiral thin wire 34m-1. The left-handed spiral thin wire 34m-2 goes over the right-handed spiral thin wire 24s-2, then goes through the right-handed spiral thin wire 24m-2, then goes over the right-handed spiral thin wire 24s-1, and then goes through the right-handed spiral thin wire 24m-1. In this manner, the knitting is performed in a plain weave shape by alternately repeating going over and going through the oblique intersection.

As illustrated in FIG. 13, on the outer peripheral surface of the winding shaft 54, the gap S1 between the right-handed spiral thin wire 24m-1 and the right-handed spiral thin wire 24m-2 corresponds to the predetermined adjacent pair interval θ1 in FIG. 9, and the gap S2 between the right-handed spiral thin wire 24m-2 and the right-handed spiral thin wire 24s-2 corresponds to the predetermined proximity interval θ2 in FIG. 9. The gap S2 between the right-handed spiral thin wire 24m-1 and the right-handed spiral thin wire 24s-1 and the gap S2 between the right-handed spiral thin wire 24m-2 and the right-handed spiral thin wire 24s-2 correspond to the predetermined proximity interval θ2 in FIG. 9.

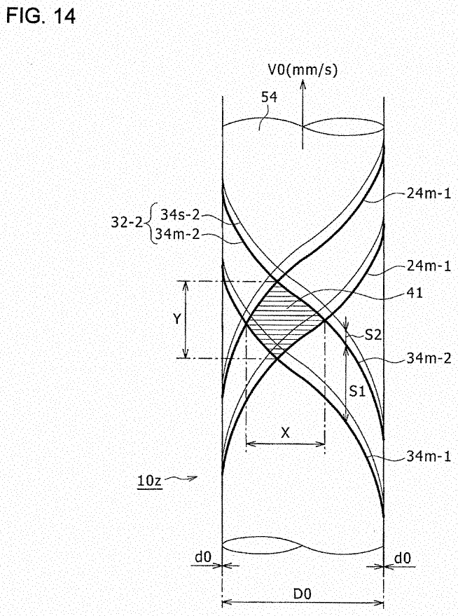

FIG. 14 is a view in which FIG. 13 is simplified and shows a stitch gap 41 formed by the right-handed spiral thin wires 24m-1 and 24m-2 and the left-handed spiral thin wires 34m-1 and 34m-2. The stitch gap 41 corresponds to the stitch gap in the stent precursor 10Z when the gap S2=0. The relationship among the size of the stitch gap 41 in the stent precursor 10Z, a revolution speed M (rotation/min) at which the carriers 70 and 71 travel on the traveling paths 60 and 61, and the setting of an axial movement speed V0 (mm/s) of the winding shaft 54 will be described using the stitch gap 41. The stitch gap 41 is larger than the predetermined stitch gap 40 in the stent 10, but the predetermined stitch gap 40 can be brought close to the size of the stitch gap 41 by shaping the predetermined proximity gap S2 to the minute gap S0. In this sense, the stitch gap 41 formed by the right-handed spiral thin wires 24m-1 and 24m-2 and the left-handed spiral thin wires 34m-1 and 34m-2 can be used as a guide of the maximum value of the predetermined stitch gap 40 of the stent 10.

With respect to the size of the stitch gap 41, a dimension along the circumferential direction on the outer peripheral surface of the winding shaft 54 is defined as X, and a dimension along the axial direction is defined as Y. When the stitch gap 41 is a square, the dimension X and the dimension Y correspond to the diagonal line length of the stitch gap 41.

The dimension X is a value obtained by dividing {π×(the outer diameter D0 of the winding shaft 54)}; that is, the outer peripheral surface length along the circumferential direction of the winding shaft 54, by (the repeating unit of the right-handed spiral thin wire pair 22 or the left-handed spiral thin wire pair 32=N) and subtracting the wire diameter d0 of the thin wires 20 and 30 from the result. That is, X=[{(π×D0)/N}−d0]. When D0 and d0 are given in mm, X is calculated in mm. The dimension X is determined regardless of the revolution speed M and the axial movement speed V0.

The dimension Y is a value obtained by subtracting the wire diameter d0 of the thin wires 20 and 30 from (a predetermined spiral pitch P of the right-handed spiral thin wire pair 22 or the left-handed spiral thin wire pair 32). The predetermined spiral pitch P is obtained by dividing the axial movement speed V0 by the revolution speed M. When the revolution speed is M (rotation/min), the axial movement speed is V0 (mm/s), and the wire diameters of the thin wires 20 and 30 are d0, Y (mm)={P (mm)−d0 (mm)}=[{60 (s)/M (rotation/min)}×V0 (mm/s)−d0 (mm)] is calculated.

Since the predetermined stitch gap 40 of the stent 10 is set to a size through which the tubular microcatheter 8 can pass, the gap shape of the predetermined stitch gap 40 is desirably square. Therefore, the gap shape of the stitch gap 41 is also desirably square, and X=Y is preferably set. From the above relationship, when the unit is mm and s (seconds), X=[{(π×D0)/N}−d0]=Y=[{(60×V0)/M}−d0] is preferably set. The above relationship becomes {(π×D0)/N}={the predetermined spiral pitch P=(60×V0)/M} by eliminating d0. Therefore, when the repeating unit N, (the predetermined stent inner diameter D0)=(the outer diameter D0 of the winding shaft 54), and the wire diameter d0 of the thin wires 20 and 30 are determined from the specification of the stent 10, the revolution speed M (rotation/min) and the axial movement speed V0 (mm/s) are set to satisfy the above relational expression.

As an example, it is assumed that the repeating unit N=6, (the predetermined stent inner diameter D0)=(the outer diameter D0 of the winding shaft 54)=3 mm, and the wire diameter d0 of the thin wires 20 and 30=0.03 mm=30 μm. In this case, X (mm)+d0 (mm)={(π×D0)/N}=1.57 mm. Since Y (mm)+d0 (mm)={(60×V0)/M}, M=0.25 rotation/s=15 rotation/min, and since {60/M (rotation/min)}=4 s, V0={1.57 (mm)/4 (s)}=0.39 (mm/s) may be set. From this, at the predetermined spiral pitch P=1.54 mm, both X (mm) and Y (mm) corresponding to the diagonal line length of the stitch gap 41 are (1.57 mm-0.03 mm)=1.54 mm.

Returning to FIG. 2 again, when the right-handed spiral thin wire 24 and the left-handed spiral thin wire 34 are knitted in a plain weave shape in a predetermined stent length while obliquely intersecting each other on the winding shaft 54 to have the appropriate stitch gap 41, the operation of the stent precursor production device 50 is stopped. Then, the knitted stent precursor 10Z is removed together with the winding shaft 54 from the stent precursor production device 50 (S22). FIGS. 13 and 14 correspond to views illustrating a part of the stent precursor 10Z on the winding shaft 54.

As described in FIGS. 13 and 14, the removed stent precursor 10Z has the predetermined proximity gap S2 and the predetermined adjacent pair gap S1. The predetermined proximity gap S2 and the predetermined adjacent pair gap S1 correspond to the predetermined proximity interval θ2 and the predetermined adjacent pair interval θ1 in the bobbin-attached carriers 74 and 75 described in FIGS. 9 and 10. Taking the example of θ2=60 degrees and θ1=about 15 degrees in angle, since the predetermined proximity interval θ2 is about (¼) of the predetermined adjacent pair interval θ1, the predetermined proximity gap S2 in the stent precursor 10Z correspondingly becomes about (¼) of the predetermined adjacent pair gap S1. In the calculation example of FIG. 13, the dimension X and the dimension Y corresponding to the diagonal line length of the stitch gap 41 correspond to the predetermined adjacent pair gap S1, and are about 1.60 mm. Assuming that the predetermined proximity gap S2 is (¼) of the predetermined adjacent pair gap S1, a gap obtained by subtracting the predetermined proximity gap S2 is (S2−S1) and is about 1.20 mm. On the other hand, the condition of the diagonal line length of the predetermined stitch gap 40 required in the stent 10 is (S1−S0)>0.78 mm when the outer diameter of the microcatheter 8 is 1.7 Fr=0.56 mm. Therefore, if the predetermined proximity gap S2 is used as the minute gap S0 of the stent 10 as it is, the microcatheter 8 can be passed through the predetermined stitch gap 40 of the stent 10, but considering manufacturing variations, there is not much room, and considerable skill and care are required when passing the microcatheter 8.

Therefore, the hand correction shaping is performed by the worker to set the predetermined proximity gap S2 in the stent precursor 10Z to the minute gap S0 that is five times or less the wire diameter of the thin wire including the contact state (S24). The hand correction shaping is performed on the stent precursor 10Z on the removed winding shaft 54 using an appropriate view expansion means or the like.

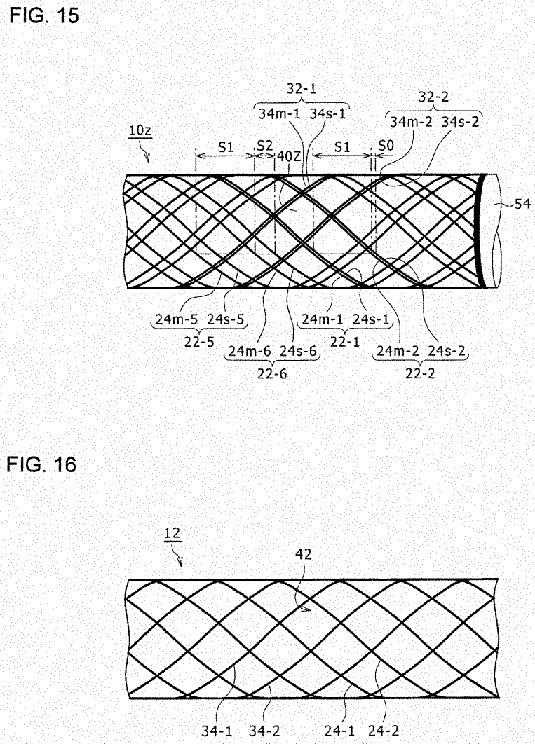

FIG. 15 is a view illustrating the stent precursor 10Z in a state where the hand correction shaping in which the predetermined proximity gap S2 is set to the minute gap S0 is performed for the right-handed spiral thin wire pairs 22-1 and 22-2 and the left-handed spiral thin wire pairs 32-1 and 32-2. The hand correction shaping has not yet been performed on the right-handed spiral thin wire pair 22 other than the right-handed spiral thin wire pairs 22-1 and 22-2 and the left-handed spiral thin wire pair 32 other than the left-handed spiral thin wire pairs 32-1 and 32-2, in a manner that the predetermined proximity gap S2 remains.

When the hand correction shaping in which the predetermined proximity gap S2 is set to the minute gap S0 is completed for all the right-handed spiral thin wire pairs 22 and all the left-handed spiral thin wire pairs 32 in the stent precursor 10Z, a stitch gap 40Z of the stent precursor 10Z becomes close to the stitch gap 41 described in FIG. 14.

The axial gap of the stitch gap 40Z of the stent precursor 10Z has a size obtained by subtracting the sum value of (the wire diameter d0 of the thin wires 20 and 30) and the minute gap S0 from the dimension Y in FIG. 14. That is, (the axial gap of the stitch gap 40Z)={Y−(the wire diameter d0 of the thin wires 20 and 30)+the minute gap S0}={(the predetermined spiral pitch P)−(2×d0)−(the minute gap S0)}.

The circumferential gap of the stitch gap 40Z of the stent precursor 10Z has a size obtained by subtracting the sum value of (the wire diameter d0 of the thin wires 20 and 30) and the minute gap S0 from the dimension X in FIG. 14. That is, (the circumferential gap of the stitch gap 40Z)={X−(the wire diameter d0 of the thin wires 20 and 30)+the minute gap}=[{(the stent inner circumferential length corresponding to the stent inner diameter)/N}−(2×d0)}−(the minute gap S0)].

Since the minute gap S0 is five times or less (the wire diameter d0 of the thin wires 20 and 30), {the sum value of (the wire diameter d0 of the thin wires 20 and 30) and the minute gap S0} is about 180 μm=0.18 mm at 6×(the wire diameter d0 of the thin wires 20 and 30) at the maximum. When X (mm)=Y (mm)=1.60 mm in FIG. 14 is used, the minimum value is (the axial gap)=(the circumferential gap)={1.60 mm-0.18 mm}=1.42 mm in the stitch gap 40Z of the stent precursor 10Z. With this size, the microcatheter 8 having an outer diameter of (1.7 Fr=0.56 mm) can be sufficiently passed with a margin.

When the hand correction shaping is completed for all the right-handed spiral thin wire pairs 22 and all the left-handed spiral thin wire pairs 32 in the stent precursor 10Z, the shape memory processing is performed by heating exceeding the transformation point of the thin wire made of the shape memory material while the stent precursor 10Z having the stitch gap 40Z is wound around the winding shaft 54 (S26). After that, the stent precursor 10Z that has been subjected to the shape memory processing is removed from the winding shaft 54 to become the stent 10. The predetermined stitch gap 40 of the stent 10 is the same as the stitch gap 40Z of the stent precursor 10Z. That is, the predetermined stitch gap 40 can pass through the microcatheter 8 having an outer diameter of 1.7 Fr=0.56 mm).

FIGS. 16 and 17 are views illustrating a case where a stent precursor is formed by a general braid knitting machine in which a gap between two adjacent right-handed spiral thin wires and a gap between two adjacent left-handed spiral thin wires 34 can be set only in the same gap, as a comparative example. Unlike the stent precursor production device 50 described above, a general braid knitting machine cannot set different gaps such as the minute gap S0 and the predetermined adjacent pair gap S1 wider than S0 with respect to a gap between two adjacent right-handed spiral thin wires 24 and a gap between two adjacent left-handed spiral thin wires 34.

FIG. 16 illustrates the stent 12 in which the stitch gap 42 having the same size as the predetermined stitch gap 40 of the stent 10 is surrounded by a total of four spiral thin wires 24 and 34 including two right-handed spiral thin wires 24 and two left-handed spiral thin wires 34 having the wire diameter d0=30 μm. Since the stitch gap 42 is the same as the predetermined stitch gap 40 of the stent 10, the microcatheter 8 having an outer diameter of (1.7 Fr=0.56 mm) can be passed through. However, since the number of the spiral thin wires 24 and 34 per unit area of the outer peripheral surface of the stent 12 is (½) of the number of the spiral thin wires 24 and 34 per unit area of the outer peripheral surface of the stent 10, the blood vessel wall expansion force of the stent 12 is about (½) of the blood vessel wall expansion force of the stent 10. If the blood vessel wall expansion force is insufficient, the stent 12 is swept away by the blood flow, and the microcatheter 8 cannot be placed at the site of the vascular aneurysm of the blood vessel.

FIG. 17 is an example of the stent 14 knitted into a hose-shaped body with the number of spiral thin wires 24 and 34 doubled per unit area of the outer peripheral surface while maintaining the wire diameter d0 of the thin wires 20 and 30 at about 30 μm. In this case, the stitch gap 44 has a size of about (¼) of the predetermined stitch gap 40 of the stent 10. Therefore, even if the blood vessel wall expansion force can be made similar to that of the stent 10, the microcatheter 8 having an outer diameter of (1.7 Fr=0.56 mm) cannot be passed through.

In addition, it is conceivable to double wind the thin wire 20 and pull out the two thin wires 20 from one thin wire supply hole 94 of the carrier 70, but the knitting yarn bobbin 80 becomes special, and entanglement and disconnection easily occur when the thin wire 20 is wound around the winding shaft 54. Therefore, it is difficult to form the right-handed spiral thin wire pair 22 and the left-handed spiral thin wire pair 32 in which the minute gap S0 is regularly disposed as in the stent 10 illustrated in FIG. 1.

On the other hand, the stent 10 can secure the size of the predetermined stitch gap 40 through which the microcatheter 8 can pass while doubling the blood vessel wall expansion force as compared with the stent 12 and has the predetermined stitch gap 40 through which the microcatheter 8 can pass as compared with the stent 14. Thus, for example, the microcatheter 8 for guiding a coil used for coil embolization is disposed within the predetermined stent inner diameter D0 of the stent 10, and the microcatheter 8 can be inserted from the predetermined stitch gap 40 toward the vascular aneurysm.

### REFERENCE SIGNS LIST

6 Microcatheter
10, 12, 14 Stent
10Z Stent precursor
20, 30 Thin wire
22, 22-1, 22-2, 22-3, 22-4, 22-5, 22-6 Right-handed spiral thin wire pair
24, 24-2, 24-2, 24m, 24m-1, 24m-2, 24s, 24s-1, 24s-2 Right-handed spiral thin wire
32, 32-1, 32-2, 32-3, 32-4, 32-5, 32-6 Left-handed spiral thin wire pair
34, 34-1, 34-2, 34m, 34m-1, 34m-2, 34s, 34s-1, 34s-2 Left-handed spiral thin wire
40 Predetermined stitch gap
40Z, 41, 42, 44 Stitch gap
50 Stent precursor production device
52 Main body housing portion
54 Winding shaft
56 Winding shaft movement mechanism
60 (One-side) traveling path
61 (Other-side) traveling path
62 (Clockwise) annular groove
63 (Counterclockwise) annular groove
64 Annular groove pair
66 Intersection position
70, 71 (Bobbin) carrier
72, 72-1, 72-2, 72-3, 72-4, 72-5, 72-6, 73, 73-1, 73-2, 73-3, 73-4, 73-5, 73-6 (Bobbin) carrier pair
74, 74m, 74m-1, 74m-2, 74m-3, 74s, 75, 75m, 75m-1, 75m-2, 75m-3, 75s, 75s-1, 75s-2, 75s-3 Bobbin-attached carrier
80 Knitting yarn bobbin
82 Bobbin base portion
84 Bobbin shaft
86 Yarn passing portion
90 Bobbin carrier driving portion
92 Thin wire lead-out hole
94 Thin wire supply hole
96 Tension weight
100 Control unit

1. A stent comprising:
with one pair consisting of two spiral thin wires in which two thin wires having superelasticity have a predetermined stent inner diameter at a predetermined spiral pitch along an axial direction, and are disposed in a minute gap that includes a contact state with each other and is five times or less a wire diameter of the thin wires,
N pairs of right-handed spiral thin wire pairs wound in a right-handed spiral shape; and
N pairs of left-handed spiral thin wire pairs wound in a left-handed spiral shape,
the stent having an axial gap of [(predetermined spiral pitch)−{2×(wire diameter of thin wire)}−(minute gap)] and a circumferential gap of [{(stent inner circumferential length corresponding to stent inner diameter)/N}−{2×(wire diameter of thin wire)}−(minute gap)] as a predetermined stitch gap formed by intersecting the right-handed spiral thin wire pair and the left-handed spiral thin wire pair in a plain weave shape.

2. The stent according to claim 1, wherein the predetermined stitch gap has a size through which a microcatheter that guides a coil placed in a cerebrovascular aneurysm can pass.

3. A stent precursor production device comprising:
a main body housing portion having a cylindrical outer shape,
a winding shaft movement mechanism that moves and drives a winding shaft at an axial movement speed along an axial direction of a central axis of the main body housing portion,
a one-side traveling path and an other-side traveling path meandering around the central axis of the main body housing portion while intersecting each other in a substantially figure-eight shape on an upper surface of the main body housing portion and making one round in a circumferential shape,
4N bobbin carriers in which there are erected a bobbin shaft rotatably supporting a knitting yarn bobbin around which a thin wire made of a shape memory alloy is wound, and a yarn passing portion that applies a predetermined tension to the thin wire pulled out from the knitting yarn bobbin and guides the thin wire to a thin wire supply hole at a predetermined height position,
with the central axis of the main body housing portion as a revolution axis,
a bobbin carrier driving portion that drives 2N bobbin carriers disposed on the one-side traveling path among the 4N bobbin carriers to travel at a revolution speed clockwise around the revolution axis, and drives other 2N bobbin carriers disposed on the other-side traveling path to travel at the revolution speed counterclockwise around the revolution axis so as not to interfere with the 2N bobbin carriers traveling clockwise at a substantially figure-eight shaped intersection position, and
a control unit that controls the revolution speed and the axial movement speed, wherein
the bobbin carrier includes 2N pairs of bobbin carrier pairs disposed at a predetermined adjacent pair interval wider than a predetermined proximity interval as one pair of the bobbin carrier pair including two bobbin carriers disposed at the predetermined proximity interval determined in advance on the one-side traveling path and the other-side traveling path.

4. A stent production method in which a stent precursor production device is used,
the stent precursor production device including
a main body housing portion having a cylindrical outer shape,
a winding shaft movement mechanism that moves and drives a winding shaft at an axial movement speed along an axial direction of a central axis of the main body housing portion,
a one-side traveling path and an other-side traveling path meandering around the central axis of the main body housing portion while intersecting each other in a substantially figure-eight shape on an upper surface of the main body housing portion and making one round in a circumferential shape,
4N bobbin carriers in which there are erected a bobbin shaft rotatably supporting a knitting yarn bobbin around which a thin wire made of a shape memory alloy is wound, and a yarn passing portion that applies a predetermined tension to the thin wire pulled out from the knitting yarn bobbin and guides the thin wire to a thin wire supply hole at a predetermined height position,
with the central axis of the main body housing portion as a revolution axis,
a bobbin carrier driving portion that drives 2N bobbin carriers disposed on the one-side traveling path among the 4N bobbin carriers to travel at a revolution speed clockwise around the revolution axis, and drives other 2N bobbin carriers disposed on the other-side traveling path to travel at the revolution speed counterclockwise around the revolution axis so as not to interfere with the 2N bobbin carriers traveling clockwise at a substantially figure-eight shaped intersection position, and
a control unit that controls the revolution speed and the axial movement speed, in which
the bobbin carrier includes 2N pairs of bobbin carrier pairs disposed at a predetermined adjacent pair interval wider than a predetermined proximity interval as one pair of the bobbin carrier pair including two bobbin carriers disposed at the predetermined proximity interval determined in advance on the one-side traveling path and the other-side traveling path,
the stent production method comprising:
disposing a knitting yarn bobbin in which the thin wire made of the shape memory alloy is wound on the bobbin shaft of each of the 4N bobbin carriers,
in each of the 4N bobbin carriers, applying a predetermined tension to the thin wire pulled out from the knitting yarn bobbin to pull out the thin wire from the thin wire supply hole at the predetermined height position,
winding each of distal ends of the pulled out 4N thin wires around the winding shaft having a predetermined outer diameter corresponding to the stent inner diameter,
moving the winding shaft at a predetermined axial movement speed along an axial direction of the revolution axis while operating a carrier driving portion at a predetermined revolution speed,
regarding N pairs of bobbin carrier pairs disposed on the one-side traveling path, driving the 2N bobbin carriers to travel clockwise at the revolution speed with respect to the revolution axis while maintaining the predetermined proximity interval and the predetermined adjacent pair interval along the one-side traveling path, and winding 2N thin wires pulled out from the thin wire supply hole of each of the 2N bobbin carriers around the winding shaft at a predetermined spiral pitch in a right-handed spiral shape to obtain 2N right-handed spiral thin wires having the stent inner diameter;
regarding N pairs of bobbin carrier pairs disposed on the other-side traveling path, driving the 2N bobbin carriers to travel counterclockwise at the revolution speed with respect to the revolution axis while maintaining the predetermined proximity interval and the predetermined adjacent pair interval along the other-side traveling path, and winding 2N thin wires pulled out from the thin wire supply hole of each of the 2N bobbin carriers around the winding shaft at a predetermined spiral pitch in a left-handed spiral shape to obtain 2N left-handed spiral thin wires having the stent inner diameter;
obliquely intersecting each of the 2N right-handed spiral thin wires and each of the 2N left-handed spiral thin wires in a plain weave shape to knit a stent precursor of a hose-shaped body while forming a diamond-like stitch gap,
removing the knitted stent precursor from the stent precursor production device together with the winding shaft,
since in a state of being wound around the winding shaft, the 2N right-handed spiral thin wires include two right-handed spiral thin wires disposed at a predetermined proximity gap corresponding to the predetermined proximity interval as one pair of the right-handed spiral thin wire pair, and N pairs of right-handed spiral thin wire pairs as a predetermined adjacent pair gap corresponding to the predetermined adjacent pair interval, and the 2N left-handed spiral thin wires include two left-handed spiral thin wires disposed at the predetermined proximity gap corresponding to the predetermined proximity interval as one pair of the left-handed spiral thin wire pair, and N pairs of left-handed spiral thin wire pairs as the predetermined adjacent pair gap corresponding to the predetermined adjacent pair interval,
performing hand correction shaping by a worker to make the predetermined proximity gap between the right-handed spiral thin wire pair and the left-handed spiral thin wire pair into a minute gap that is five times or less the wire diameter of the thin wire including a contact state; and
with a predetermined stitch gap formed by intersecting the right-handed spiral thin wire pair and the left-handed spiral thin wire pair in the plain weave shape as an axial gap of [(predetermined spiral pitch)−{2×(wire diameter of thin wire)}−(minute gap)] and a circumferential gap of [{(stent inner circumferential length corresponding to stent inner diameter)/N}−{2×(wire diameter of thin wire)}−(minute gap)],
performing shape memory processing by heating exceeding a transformation point of the thin wire made of a shape memory material in a state where the stent precursor having the predetermined stitch gap is wound around the winding shaft.
